(19) United States
(12) Patent Application Publication
(10) Pub. No.: US 2026/0021163 A1
(43) Pub. Date: Jan. 22, 2026

(54) METHODS OF TREATING MALIGNANT GLIOMAS

(71) Applicant: Targepeutics, Inc., Hummelstown, PA (US)

(72) Inventors: Randy Schrecengost, Lancaster, PA (US); Silvan B Lutkewitte, III, Hershey, PA (US)

(21) Appl. No.: 18/993,145

(22) PCT Filed: Jun. 21, 2023
(86) PCT No.: PCT/US2023/068842

§ 371 Date: Jan. 10, 2025

Related U.S. Application Data
(60) Provisional application No. 63/368,283, filed on Jul. 13, 2022.

(51) Int. Cl. A61K 38/20; A61K 38/16; A61P 35/00; G01N 33/68

(52) U.S. Cl. CPC A61K 38/2086; A61K 38/164; A61P 35/00; G01N 33/6869; G01N 33/6893

(57) ABSTRACT

Disclosed are methods of treating cancer in a subject comprising (i) administering a pharmaceutical composition comprising a therapeutically effective amount of a mutagenized IL 13 moiety (mIL 13); and then (ii) delivering a radiation therapy.

### CROSS-REFERENCE TO RELATED APPLICATIONS

This application claims the benefit of U.S. Provisional Patent Application Ser. No. 63/368,283 filed on Jul. 13, 2022, incorporated herein by reference.

### GOVERNMENT CONTRACT

This disclosure was made with Government support under Government Contract No. CA275560 awarded by the National Institutes of Health. The United States Government has certain rights in the invention.

### FIELD

The present disclosure relates to cancer therapy and more specifically relates to treatment of malignant gliomas.

### BACKGROUND

Prognosis for malignant gliomas remains poor due to the lack of effective therapeutic treatments. The limited effectiveness of the current therapeutic modalities for certain cancers highlights the necessity for the development of effective and novel treatments for patients suffering from these diseases.

### SUMMARY

Disclosed herein are methods of treating cancer in a subject comprising (i) administering a pharmaceutical composition comprising a therapeutically effective amount of a mutagenized IL13 moiety (mIL13); and (ii) delivering a radiation therapy.

### BRIEF DESCRIPTION OF THE DRAWINGS

FIG. 1 shows results of immunoblotting assays of DIPG cell lines treated with IL13Rα2, IL13Rα1, and GAPDH antibodies.

FIG. 2 shows a graph of tumor cell survival as a function of cmIL13 dosage.

FIG. 3 shows a graph of total SF8628 DIPG cells as a function of time following (1) vehicle, (2) 2 Gy of radiation treatment, (3) administration of 1 ng/ml of cmIL13, or (4) administration of 1 ng/ml of cmIL13 followed by 2 Gy of radiation treatment.

FIG. 4 shows a graph of total U87-MG GBM cells as a function of time following (1) vehicle, (2) 10 Gy of radiation treatment, (3) administration of 10 ng/mL of cmIL13, or (4) administration of 10 ng/ml of cmIL13 followed by 10 Gy of radiation treatment.

FIG. 5 shows a bar graph of relative DIPG IV cell viability at 96 hours and 120 hours post-treatment for cells treated with (1) a vehicle, (2) 2 Gy of radiation, (3) 0.25 ng/ml cmIL13, or (4) 0.25 ng/mL cmIL13 followed by 2 Gy of radiation.

FIG. 6 shows an immunoblotting assay of SF8628 cells of cleaved caspase 3, cleaved caspase 7, and GAPDH.

FIG. 7 shows a graph of survival fraction of SF8628 DIPG cells as a function of radiation strength for (1) cells treated with radiation alone and (2) cells treated with 0.5 ng/ml cmIL13 followed by radiation.

FIG. 8 shows a graph of percent survival of subjects when treated with (1) vehicle and sham radiation, (2) 0.005 mg/ml cmIL13 and sham radiation, (3) vehicle and radiation therapy, and (4) 0.005 mg/ml cmIL13 and radiation therapy.

FIG. 9 shows SEQ. ID. NOS: 1-24.

### REFERENCE TO AN ELECTRONIC SEQUENCE LISTING

The contents of the electronic sequence listing (Targepeutics Sequence Listing.xml; Size: 27,000 bytes; and Date of Creation: Jul. 10, 2022) is herein incorporated by reference in its entirety.

### DETAILED DESCRIPTION

Unless defined otherwise, all technical and scientific terms used herein have the same meaning as commonly understood by one of ordinary skill in the art to which the disclosure pertains. Although any methods and materials similar or equivalent to those described herein can be used in practice for testing of the present disclosure, the preferred materials and methods are described herein.

For purposes of the following description, it is to be understood that the disclosure may assume various alternative variations and step sequences, except where expressly specified to the contrary. Moreover, other than in any operating examples, or where otherwise indicated, all numbers such as those expressing values, amounts, percentages, ranges, subranges, and fractions may be read as if prefaced by the word “about,” even if the term does not expressly appear. Accordingly, unless indicated to the contrary, the numerical parameters set forth in the following specification and attached claims are approximations that may vary depending upon the desired results to be obtained by the present disclosure. At the very least, and not as an attempt to limit the application of the doctrine of equivalents to the scope of the claims, each numerical parameter should at least be construed in light of the number of significant digits and by applying ordinary rounding techniques. Where a closed or open-ended numerical range is described herein, all numbers, values, amounts, percentages, subranges, and fractions within or encompassed by the numerical range are to be considered as being specifically included in and belonging to the original disclosure of this application as if these numbers, values, amounts, percentages, subranges, and fractions had been explicitly written out in their entirety.

Notwithstanding that the numerical ranges and parameters that set forth the broad scope of the disclosure are approximations, the numerical values set forth in the specific examples are reported as precisely as possible. Any numerical value, however, inherently contains certain errors necessarily resulting from the standard variation found in their respective testing measurements.

As used herein, unless indicated otherwise, a plural term can encompass its singular counterpart and vice versa. For example, although reference is made herein to “a” catheter, or “an” IL13, any combination (i.e., a plurality) of these components can be used. In addition, in this application, the use of “or” means “and/or” unless specifically stated otherwise, even though “and/or” may be explicitly used in certain circumstances.

As used herein, “including,” “containing” and like terms are understood in the context of this application to be synonymous with “comprising” and are therefore open-ended and do not exclude the presence of additional undescribed and/or unrecited elements, materials, ingredients, and/or method steps.

As used herein, “consisting of” is understood in the context of this application to exclude the presence of any unspecified element, ingredient, and/or method step.

As used herein, “consisting essentially of” is understood in the context of this application to include the specified elements, materials, ingredients, and/or method steps “and those that do not materially affect the basic and novel characteristic(s)” of what is being described.

As used herein, the terms “patient,” “subject,” “individual,” and the like are used interchangeably herein and mean animals amenable to the methods of treatment described herein, including mammals, including a human, a canine, a feline, a bovine, an equine, a porcine, a primate, and/or a rodent.

As used herein, “administering” or “administration” of an amount (e.g., a dose) of a composition may be done by the subject himself/herself or another subject (e.g., a medical professional, a caretaker, or a family member). The composition may be provided by the subject or the administrator for the subject along with instructions for the administration of the composition (e.g., written instructions on the label of a container containing the composition).

As used herein, “disorder,” “disease,” and “illness” are used interchangeably and refer to a condition in a subject that negatively impacts the health of the subject.

As used herein, “treat,” “treatment,” or “treating” means treatment of a disease or disorder, as defined herein, in a subject, including: (1) inhibiting a disease or disorder; (2) arresting the development of a disease or disorder; (3) slowing progression of the disease or disorder; and/or (4) inhibiting, relieving, or slowing progression of one or more symptoms of the disease or disorder. A disease, disorder, or illness is “treated” if the subject experiences a reduction in the severity and/or frequency of the disease, disorder, and/or illness or a symptom associated therewith.

As used herein, “cancer” refers to a disease characterized by the abnormal growth of aberrant cells. Cancer cells can spread locally or through the bloodstream and lymphatic system to other parts of the body. Examples of various cancers include those brain cancers in which tissue cells express IL13Rα2, including but not limited to gliomas and the like.

As used herein, “glioma” refers to a tumor that originates in glial cells of the brain or spinal cord.

As used herein, a “therapeutically effective amount” is defined as an amount required to ameliorate the symptoms of a disease in a treated patient relative to an untreated patient. The effective amount of active compound(s) used to therapeutically treat a disease varies depending upon the manner of administration, as well as the age, body weight, and general health of the subject.

As used herein, “composition” refers to a solution or dispersion.

As used herein, “pharmaceutical composition” refers to any chemical or biological composition, material, agent, or the like that is capable of inducing a therapeutic effect when properly administered to a subject, including the composition, material, agent, or the like in an inactive form and active metabolites thereof, where such active metabolites may be formed in vivo.

As used herein, “radiation therapy” refers to a method of treating a patient comprising internal or external radiation treatment.

As used herein, “radiation treatment” refers to delivery of radioactive energy, such as electrons, protons, or heavy ions, into a target volume, such as a tumor or lesion, within a patient.

As used herein, “measuring” or “measurement” or “detecting” or “detection” means assessing the presence, absence, quantity, or amount (which can be an effective amount) of either a given substance within a clinical or subject-driven sample, including the derivation of qualitative or quantitative levels of such substance, or otherwise evaluating the values or categorization of a subject's clinical parameters.

As used herein, “sample” or “biological sample” refers to a biological material isolated from an individual, such as a liquid or solid biological sample collected via a biopsy (i.e., a “liquid biopsy” or a “solid biopsy”). A liquid biopsy may comprise, for example, blood, plasma, saliva, urine, cerebral spinal fluid, and/or other body fluid. The liquid biopsy may contain extracellular vesicles and/or cell-free genetic material. A solid biopsy may comprise, for example, an organ and/or a tissue, such as a tumor. The biological sample may contain any biological material suitable for detecting the desired biomarkers and may comprise cellular and/or non-cellular material obtained from the individual.

As used herein, “control” refers to any experimental condition that is either not treated at all or is treated by a method that is not disclosed herein. For example, a sample or subject that is treated with radiation therapy alone is a control. In another example, a sample or subject that is treated with a pharmaceutical composition comprising a therapeutically effective amount of a mutagenized IL13 alone is a control.

As used herein, “interleukin-13” or “IL. 13” refers to any wild-type, i.e., native, IL13 from any vertebrate source, including mammals, such as primates and rodents, unless indicated to the contrary, and includes unprocessed IL13 and any form of IL13 that results from processing in a cell and any naturally occurring variants of IL13, such as splice or allelic variants. The amino acid sequence of an exemplary human IL13 is shown in SEQ ID NO:1. An amino acid sequence of a second exemplary human IL13 is shown in SEQ ID NO:2.

A “mutation” in a polypeptide is meant to encompass proteins having any amino acid substitutions, deletions (e.g., a truncated version of the protein, such as a polypeptide), insertions, and/or modifications, such as by glycosylation, phosphorylation, acetylation, myristoylation, prenylation, palmitoylation, amidation, and the like. In an example, a “mutated IL13” or a “mutagenized IL13” refers to an IL13 in which one or more of the amino acids differs from the corresponding amino acids in the wild-type form of IL13. Mutated IL13 and/or mutagenized IL13 may be derived from the wild-type form of IL13 found in humans, non-human primates, rats, murine, porcine, bovine, canine, and the like. A mutated IL13 and/or mutagenized IL13 may be referred to herein as “mIL13.” mIL13 may be connected to a cytotoxin (“cmIL13”), as further discussed herein. As used herein, “mIL13” includes “cmIL13.”

As used herein, the term “IL13 receptor” or “IL13R” refers to a receptor that binds IL13.

As used herein, the term “IL13 receptor α 2” or “IL13Rα2” refers to the monomeric IL13 receptor that is expressed on the surface of specific cell subsets and that binds IL13 or any homolog thereof, such as mIL13 or is contained in extracellular vesicles and that binds IL13 independent of IL4.

As used herein, “affinity” refers to the strength of the total of non-covalent interactions between a single binding site of a receptor and a ligand. The affinity of a receptor for a ligand can be represented by the dissociation constant (K_{D}), which is the ratio of dissociation and association rate constants, K_{off} and Kₒₙ, respectively. Affinity can be measured by methods known to those of skill in the art.

“Increased binding” refers to binding levels of an mIL13 which are at least 10% or more, such as 20%, 30%, 40%, 50%, 60%, 70%, 80%, 90% higher or more, or 1-fold, 2-fold, 5-fold, 10-fold, 20-fold, 100-fold, 1000-fold higher or more, and any and whole or partial increments therebetween, than a wild-type IL13.

“Decreased binding” refers to binding levels of an mIL13 which are at least 10% or less, such as 20%, 30%, 40%, 50%, 60%, 70%, 80%, 90% lower or less, or 1-fold, 2-fold, 5-fold, 10-fold, 20-fold, 100-fold, 1000-fold lower or less, and any and whole or partial increments therebetween, than a wild-type IL13.

As used herein, “fusion” or “fused” is defined as the linking of one gene to a second gene pre-translation.

As used herein, “determining the level of marker (or biomarker) expression” is meant as an assessment of the degree of expression or presence of a marker in a sample at the nucleic acid or protein level, using technology available to the skilled artisan to detect a sufficient portion of any marker expression product, such as “determining the level of IL13Rα2.”

As used herein, the “level” of one or more marker (or biomarker) means the absolute or relative amount or concentration of the marker (or biomarker) in the sample.

As used herein, “ng/ml” means mass of protein in a volume of 1 ml.

The present disclosure is directed to a method of treating cancer in a subject comprising, consisting essentially of, or consisting of administering a pharmaceutical composition comprising a therapeutically effective amount of a mutagenized IL13 moiety (mIL13); and then delivering a radiation therapy.

The pharmaceutical composition may comprise, consist essentially of, or consist of an mIL13. The mIL13 may be engineered to have increased affinity for IL13Rα2 compared to wild-type human IL13. The affinity of the mIL13 for IL13Rα2 may be increased compared to a wild-type IL13, such as a 2-fold increase, a 3-fold increase, a 5-fold increase, a 10-fold increase, a 100-fold increase, a 1000-fold increase, or more of kinetic K_{D}.

The mIL13 also may be engineered to have decreased affinity for the interleukin 13 receptor α 1 (IL13Rα1) compared to wild-type human IL13. For example, the mIL13 may have a reduced affinity to the IL13Rα1 compared to a wild-type IL13, for example, a 2-fold decrease, a 3-fold decrease, a 5-fold decrease, a 10-fold decrease, a 100-fold decrease, or more of kinetic K_{D} while at least retaining binding or activation of the IL13Rα2.

In examples, the mIL13 may be produced by cDNA mutagenesis, DNA synthesis, peptide/protein synthesis, or any method known to those of skill in the art.

The mIL13 may be a full-length IL13 molecule, such as a human full-length IL13 molecule. In examples, the mIL13 may comprise amino acid changes relative to wild-type IL13 at a position corresponding to residue 13 of the human IL13 (SEQ ID NO:1), at a position corresponding to residue 66 of SEQ ID NO: 1, at a position corresponding to residue 69 of SEQ ID NO: 1, and/or at a position corresponding to residue 105 of SEQ ID NO:1. The mIL13 may comprise a substitution to the glutamic acid at a position 13 of the human IL13 (SEQ ID NO:1). For example, lysine may be substituted for the glutamic acid at a position 13. The mIL13 may comprise a substitution to the arginine at position 66 of the human IL13 (SEQ ID NO:1). For example, aspartic acid may be substituted for the arginine at position 66. The mIL13 may comprise a substitution to the serine at position 69 of the human IL13 (SEQ ID NO:1). Aspartic acid may be substituted for the serine at position 69. The mIL13 may comprise a substitution of the lysine at position 105 of the human IL13 (SEQ ID NO:1). Arginine may be substituted for the lysine at position 105. The mIL13 may comprise changes at positions E13, R66, S69, and/or K105. The mIL13 may be designated IL13.E13K.R66D.S69D.K105R. In an example, the mIL13 may comprise the amino acid sequence set forth in SEQ ID NO:3. In other examples, the mIL13 may comprise any of SEQ ID NOS: 4 to 22, or may comprise a sequence sharing at least 50, 60, 70, 80, 85, 90, 95, or 99% homology with any of SEQ ID NOS: 1 to 24, or may comprise a sequence that differs by no more than 1, 2, 3, 4, 5, 6, 7, 8, 9, 10, 15, or 20 residues from any of SEQ ID NOS: 1 to 24.

The mIL13 and analogs thereof provide for selective binding to IL13 receptors. The mIL13 may be a highly selective antagonist and/or selective agonist of wild-type IL13 activity. The mIL13 is engineered to have one or more of the following properties: (a) altered affinity (either decreased affinity or increased affinity) for IL13Rα2 relative to wild-type human IL13; (b) altered affinity (either decreased affinity or increased affinity) for IL13Rα1 relative to wild-type human IL13; and/or (c) a disruption in the binding sight for IL4Rα1. The mIL13 may comprise amino acid modifications made at one or more of the amino acids within the set of contact residues that interact with IL13Rα1, IL13Rα2, or IL4Rα1, which residues include, for example L10, R11, 114, E12, V18, R65, R86, D87, T88, K89, L10I, K104, K105, F107, and R108 (for reference purposes the sequence of wild-type human IL13 is provided as SEQ. ID. NO: 1, to which the numbering of amino acids refers). The mIL13 may comprise modifications at two or more, three or more, four or more, five or more, and not more than 14 amino acids within the combined set of contact residues defined herein.

The mIL13 may comprise one or more of the following amino acid substitutions: (1) L10F; L10I; L10V; L10A; L10D; L10T; L10H; (2) R11S; R11N; R11H; R11L; R11I; (3) I14L; I14F; I14V; I14M; (4) V18L; V18F; V18I; (5) E12A; (6) R65D; (7) R86K; R86T; R86M; (8) D87E; D87K; D87R; D87G; (9) T881; T88K; T88R; (10) K89R; K89T; K89M; (11) L101F; L101I; L101Y; L101H; L101N; (12) K104R; K104T; K104M; (13) K105T; K105A; K105R; K105E; (14) F107L; F107I; F107V; F107M; and (15) R108K; R108T; R108M. These substitutions cause an altered affinity for one or both of IL13Rα1 and IL13Rα2. The mIL13 may comprise modified residues at two or more, three or more, four or more, five or more, and not more than 14 amino acids within the combined set of contact residues defined herein.

The term “homology” and “homologous” refers to the subunit sequence identity between two molecules, such as two protein or peptide molecules. When a subunit position in both of the molecules is occupied by the same subunit, then the molecules are homologous at that position. The homology between two sequences is a direct function of the number of matching or homologous positions. For example, if half of the positions in two sequences are homologous, then the two sequences are 50% homologous and if 70% of the positions (i.e., 7 of 10) are matched or homologous, then the two sequences are 70% homologous. Homologs of the present disclosure can be the result of natural allelic variation, including natural mutation. Homologs of the present disclosure can be the result of natural allelic variation, including natural mutation. Homologs of the present disclosure can also be produced using techniques known in the art, including direct modifications to the protein, using, for example, recombinant DNA techniques to effect random or targeted mutagenesis.

The mIL13 may be complexed with an extracellular vesicle (EV) to form an extracellular vesicle complex (EV complex). The extracellular vesicle may be obtained from a particular type of biological sample and/or may be derived from a particular type of cell, such as glioma stem cells. In examples, the glioma stem cells may be mesenchymal glioma stem cells or proneural glioma stem cells. The extracellular vesicle may comprise an exosome, such as a tumor-associated exosome. The extracellular vesicles may be purified or concentrated from a biological sample using differential centrifugation, ultracentrifugation, and/or other methods known to those of skill in the art.

Subpopulations of extracellular vesicles may be isolated using a biological marker. The biological marker may be a receptor, such as a tumor-associated receptor. The tumor-associated receptor may be IL13Rα2. Each exosome may, for example, express 1, 2, 5. 10, 15, 20, 25, 50, 100, 250, 500, 1000, or more biological markers.

A cytotoxin may be fused to one of the mIL13 disclosed herein (“cmIL13”). For example, the fusion may occur by 1-ethyl-3-(3-dimethylaminopropyl) carbodiimide (“EDC”) chemistry or purified gel filtration.

The cytotoxin may comprise, consist essentially of, or consist of a bacterial-derived toxin. The bacterial-derived toxin may comprise, for example, Pseudomonas exotoxin A.

The pharmaceutical composition may be administered by a catheter. For example, the pharmaceutical composition may be administered by convection enhanced delivery (CED). As used herein, “convection enhanced delivery” or “CED” refers to a method of drug administration wherein a pressure gradient is generated at the tip of an infusion catheter for direct delivery of therapeutics through the interstitial spaces of the central nervous system to the structure to be treated, e.g., into a resection cavity or into an intact tumor. The pressure gradient may be a positive pressure gradient, meaning that the pharmaceutical composition may be administered under positive pressure. The pharmaceutical composition also may be administered by CED at a constant pressure. Any suitable catheter known in the art may be used. The pharmaceutical composition may be administered by convective infusion at a specified flow rate controlled by an external syringe pump. The pharmaceutical composition may be administered at a flow rate of up to 2 mL/hour. The method comprises positioning the tip of a catheter within the area to be treated. An external pump may be connected to the catheter, which supplies a composition comprising a therapeutically effective dose of a therapeutic agent, e.g., mIL13, while maintaining a positive pressure gradient through delivery. Administration of mIL13 may or may not occur with co-infusion of an imaging component to serve as a surrogate marker for drug delivery, e.g., gadolinium or gadolinium-DTPA.

The pharmaceutical composition may be administered acutely. As used herein, “acute administration” and the like refers to an in-patient procedure in which the pharmaceutical composition is administered to the patient by a healthcare professional. For example, administration of the pharmaceutical composition may occur over a period not to exceed one week. For example, administration of the pharmaceutical composition may occur over a period of at least 4 hours. In other examples, administration of the pharmaceutical composition may occur over a period of no more than 96 hours. Administration of the pharmaceutical composition may occur over a period of 4 hours to 96 hours.

The pharmaceutical composition comprising mIL13 may be administered wherein the dose of mIL13 is from at least 0.03 μg/mL to no greater than 1 μg/mL.

The method of treating cancer in a subject disclosed herein further comprises, consists essentially of, or consists of delivering a radiation therapy. The radiation therapy may be any suitable radiation therapy known to those of skill in the art. In examples, the radiation therapy may be fractionated radiation therapy.

The radiation therapy may be administered following administration of the pharmaceutical composition comprising an mIL13. In addition to being administered prior to radiation therapy, the pharmaceutical composition may also be administered concurrently with the radiation therapy.

Any type of tumor comprising cells that express IL13Rα2 may be treated with the methods disclosed herein. For example, the methods disclosed herein may be used to treat cancers of the brain and/or central nervous system (CNS), such as malignant gliomas. Examples of malignant gliomas that may be treated with the method disclosed herein include but are not limited to an adult glioblastoma, a pediatric glioblastoma, an anaplastic astrocytoma, such as a diffuse midline glioma, an anaplastic oligodendroglioma, an anaplastic oligoastrocytoma, an anaplastic ependymoma, and/or an anaplastic ganglioma. In other examples, the methods disclosed herein may be used to treat soft-tissue sarcomas.

Prior to administration of the mIL13, expression of IL13Rα2 may be detected in a sample of the tumor. In examples, the sample of the tumor may be purified prior to detection of expression of IL13Rα2. Detecting expression of IL13Rα2 may be detected by mass spectrometry, analytical assay, immunostaining, and/or sequencing. In examples, the analytical assay may comprise an enzyme-linked immunosorbent assay (ELISA). In other examples, the immunostaining may comprise immunohistochemistry (IHC) and/or fluorescent cytochemistry. In other examples, the sequencing comprises whole genome sequencing, exome sequencing, proteomic sequencing, and/or RNA sequencing. Expression of the IL13Rα2 by the tumor is indicative that the tumor will be responsive to treatment by the mIL13.

It has been surprisingly discovered that treatment of a tumor expressing IL13Rα2 with a pharmaceutical composition comprising an mIL13 prior to administration of radiation therapy enhances the anti-tumor effect of the treatments alone.

It has also been surprisingly discovered that treatment of a tumor expressing IL13Rα2 with a pharmaceutical composition comprising an mIL13 prior to administration of radiation therapy decreases the strength of radiation necessary in order to achieve the same level of tumor cell death as compared to treatment with radiation therapy alone.

It will be appreciated by those skilled in the art that numerous modifications and variations are possible in light of the above disclosure without departing from the broad inventive concepts described and exemplified herein. Accordingly, it is therefore understood that the foregoing disclosure is merely illustrative of various exemplary aspects of this application and that numerous modifications and variations can be readily made by skilled artisans which are within the spirit and scope of this application and the accompanying claims.

### EXAMPLES

### Example 1: CmIL13 Cytotoxicity Correlates with IL13Rα2 Expression

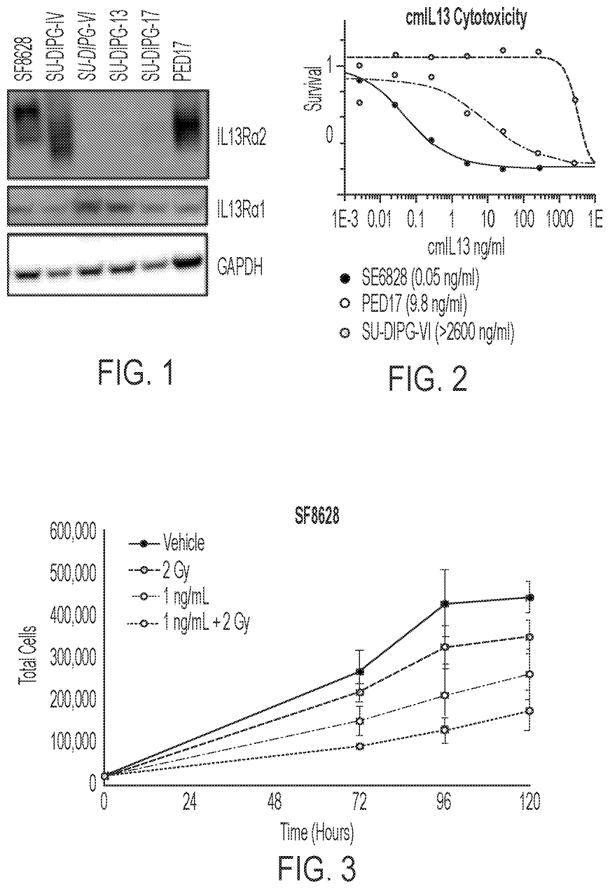

Selection of DIPG cell lines were harvested and lysates were processed for immunoblotting with IL13Rα2, IL13Rα1, and GAPDH antibodies. FIG. 1 shows the immunoblotting assays.

Cell lines with varying IL13Rα2 expression levels were tested in a cell potency assay. Varying concentrations of a mutagenized IL13 fused to a cytotoxin (cmIL13) were added to plated cells for 72 hours, after which time cell viability was determined by MTS assay. As shown in FIG. 2, cell lines with high IL13Rα2 displayed sensitivity (SF6828 IC50=0.05 ng/ml; PED17 IC50=9.8 ng/ml) while cells lacking IL13Rα2 were insensitive to the highest treatment (SU-DIPG-IV IC50>2600 ng/ml).

### Example 2: CmIL13 Administration Prior to Radiation Enhances Anti-Tumor Effect on DIPG and GBM Cells In Vitro

On Day 1, DIPG and GBM cells were seeded onto well plates and allowed to adhere to the plates. 25,000 cells of SF8628 (DIPG cell line), 50,000 cells of U87-MG (GBM cell line), and 50,000 cells of DIPG IV (DIPG cell line) were seeded onto 6-well plates, respectively. SF8628 cells were treated in triplicate with 1 ng/mL of cmIL13, U87-MG cells were treated in triplicate with 50 ng/ml of cmIL13, and DIPG IV cells were treated in triplicate with 0.25 ng/mL. All plates were then placed in an incubator. The cells were irradiated with a single dose of X-ray radiation (2 Gy for the SF8628 cells, 10 Gy for the U87-MG cells, and 2 Gy for DIPG IV cells) 16 hours after cmIL13 administration. The plates were returned to the incubator and the cell number was counted by hemacytometer 72 hours, 96 hours, and 120 hours after radiation.

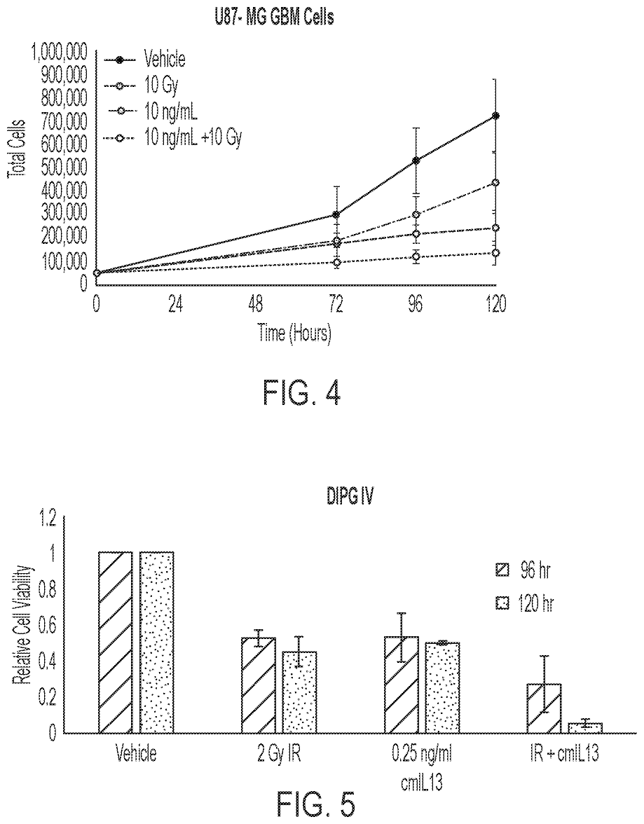

FIG. 3 shows the results for the SF6828 DIPG cell assays, FIG. 4 shows the results for the U87-MG GBM assays, and FIG. 5 shows the results of the DIPG IV assays. All tested cell types experienced lower total cell counts when treated with cmIL13 followed by radiation therapy when compared to cells that were not treated or cells treated with only one of cmIL13 or radiation therapy. When radiation therapy follows cmIL13 treatment, an additive effect was noted, resulting in the lowest total cell count of all treatments examined. These results also demonstrate that cmIL13 does not negatively impact the effects of radiation.

### Example 3: CmIL13 and Radiation Combination Induces Enhanced Caspase-Mediated Cell Death

100,000 cells of SF8628 were seeded onto 5 individual 60 mm plates. The cells of each plate were treated in one of five conditions-(1) vehicle; (2) treatment with vehicle followed by a single dose of 4 Gy X-ray radiation 24 hours after vehicle administration; (3) treatment with 1 ng/mL of cmIL13: (4) treatment with 2.5 ng/mL cmIL13; or (5) treatment with 1 ng/mL cmIL13 followed by a single dose of 4 Gy X-ray radiation 24 hours after cmIL13 administration. SF8628 cells were treated in triplicate with 1 ng/ml of cmIL13. All plates were then placed in an incubator for 72 hours following radiation treatment.

The tumor cells were lysed in Triton X-100 lysis buffer containing protease inhibitors and sonicated. Collected protein lysates were stored at −20° C. Protein concentrations were determined using the Pierce BCA Protein Assay Kit (Cat #23227; Thermo Fisher Scientific). 30 μg of total protein was size fractioned by 12.5% SDS-PAGE. Electrophoresis-separated proteins were electrically transferred to a polyvinylidene difluoride (PVDF) membrane, washed in PBST buffer, and blocked in 2% fat-free milk for 1 h at room temperature, then incubated with primary antibodies at 4° C. overnight. Following primary antibody blotting, specific signal was detected with species-appropriate peroxidase-conjugated secondary antibody (Thermo Fisher Scientific) using SuperSignal West Pico PLUS Chemiluminescent Substrate (Cat #34580; Thermo Fisher Scientific) and imaged using a BioRad GelDoc XR imaging system (BioRad, USA). Details regarding antibodies used for Western blots can be found in Table 1.

TABLE 1

| Antibodies | Source | Identifier | Dilution |
|---|---|---|---|
| Cleaved Caspase 3 | Cell | 9664S | 1:1000 |
| | Signaling | | |
| Cleaved Caspase 7 | Cell | 8438 | 1:1000 |
| | Signaling | | |
| GAPDH | Abclonal | AC035 | 1:10000 |

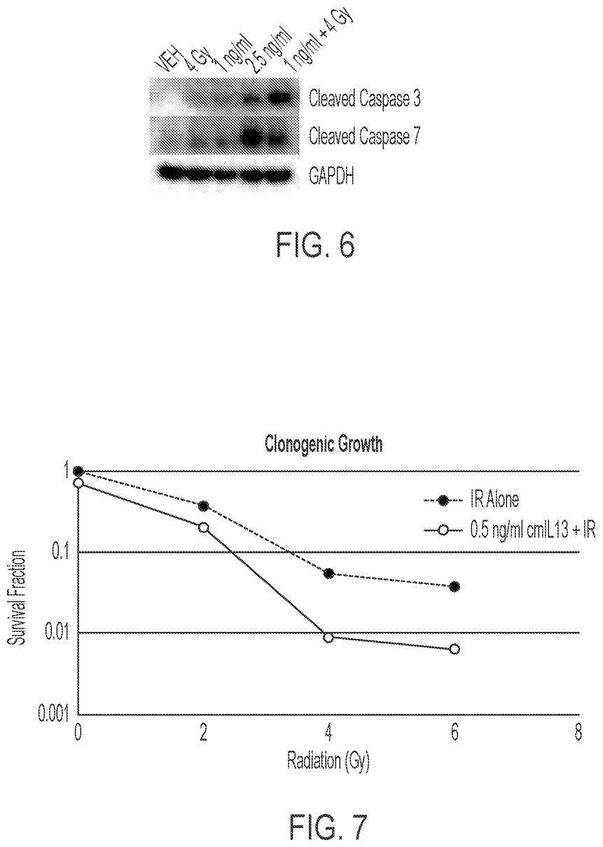

FIG. 6 provides data collected from the Western blot. As shown in FIG. 6, tumor cells treated with a combination therapy of 1 ng/mL of cmIL13 followed by 4 Gy radiation exhibited intense staining for the apoptosis markers cleaved caspase 3 and cleaved caspase 7. These data indicate that treatment of cells with cmIL13 prior to radiation therapy led to a substantial increase in cell death compared to the other treatment conditions.

### Example 4: CmIL13 Plus Radiation Inhibits Colony Formation of DIPG Cells

SF8628 cells were plated in 6-well plates at a density empirically determined based on the plating efficiency for each radiation dose that results in cell colonies not overlapping after fourteen days which was: 0 Gy—500 cells per well; 2 Gy—1000 cells per well; 4 Gy—3000 cells per well; and 6 Gy—8000 cells per well. The SF8628 cells were treated with either a vehicle or 0.5 ng/mL cmIL13. 24 hours following cmIL13 or vehicle treatment, the SF8628 cells were treated with either 0 Gy, 2 Gy, 4 Gy, or 6 Gy radiation and incubated for fourteen days. At the end of the incubation, cells were fixed with ice-cold methanol and stained with crystal violet. Colonies with more than 50 cells were counted for all conditions. As shown in FIG. 7, the combination treatment of cmIL13 prior to radiation substantially decreased the survival fraction of SF8628 cells when compared to treatment with radiation alone. Therefore, treatment with cmIL13 prior to radiation resulted in an enhanced anti-tumor effect.

### Example 5: CmIL13 and Radiation Treatment of SU-DIPG-XVII Malignant Glioma in Mice

Tumor Preparation: SU-DIPG-XVII cells were injected directly into the pons of the mice. Tumor presence was determined by MRI. Animals with confirmed tumors were divided into four treatment groups: (1) vehicle pump+sham radiation; (2) cmIL13 pump+sham radiation; (3) vehicle pump+radiation; or (4) cmIL13 pump+radiation.

cmIL13 Administration: ALZET® osmotic pumps (Model 2001; infusion rate: 1 μL/hr) were primed per the manufacturer's instructions. Each pump received a solution of 0.005 mg/mL cmIL13 (total dose of 1 μg) or vehicle. The pump was connected to a catheter primed with the drug solution and connected to a 30-gauge cannula (5 mm projection below the pedestal).

Animals underwent Alzet pump implantation surgery on approximately day 85 post-tumor engraftment. Animals were anesthetized and a midline incision was made to expose the skull. A burr hole was drilled using a Foredom MH-130 portable drill (Foredom) at the coordinates 1 mm posterior to the lamboid suture, 1 mm lateral to the mid-sagittal plane, and 4.2 mm in depth. A subcutaneous pocket was then created between the scapulae using a hemostat at the posterior end of the incision. The ALZET® pump connected to the infusion cannula via a vinyl catheter was inserted into the subcutaneous pocket. Dental glue primer (Kulzer) was applied to the skull surrounding the burr hole with a 0.3 mL insulin syringe (BD) and the primer was activated with UV light. The animals were each placed in a stereotactic frame and a stereotactic insertion arm was used to insert the ALZET® cannula (Plastics One, 7 mm below the pedestal). Dental glue (Ivoclar Vivadent) was used to affix the guide cannula to the skull. The skin incision was closed with wound clips. cmIL13 or vehicle was then administered continuously for a period of seven days at an infusion rate of 1 μL/hour at a total dose of 1 μg for treatment groups receiving cmIL13. Eight days after pump implantation the ALZET pump was removed. Animals were anesthetized and a small incision was made between the scapulae near the pump. The catheter was disconnected and sealed with bone wax and the pump was removed. The incision was closed with 4.0 vicryl suture (Ethicon).

Radiation Treatment: Two days after the cmIL13 or vehicle administration was initiated, animals in treatment groups (1) and (2) were exposed to sham radiation and treatment groups (3) and (4) were exposed to 2 Gy ionizing radiation for five consecutive days using image-guided focal radiation. The radiation was delivered by using an X-RAD SmART irradiator that allowed image-guided, focal delivery with shielding. To deliver the radiation, mice were anesthetized under isoflurane and cranially fixed on the stereotactic stage using the bite block without pins.

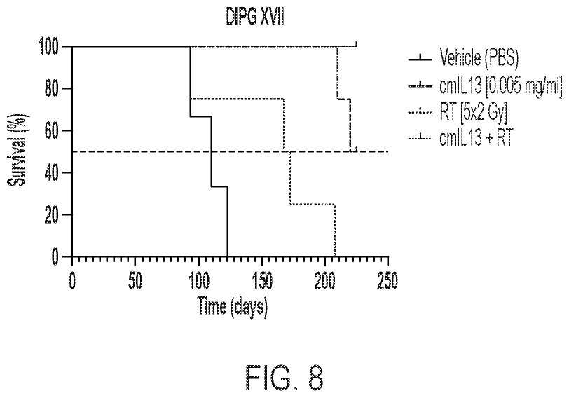

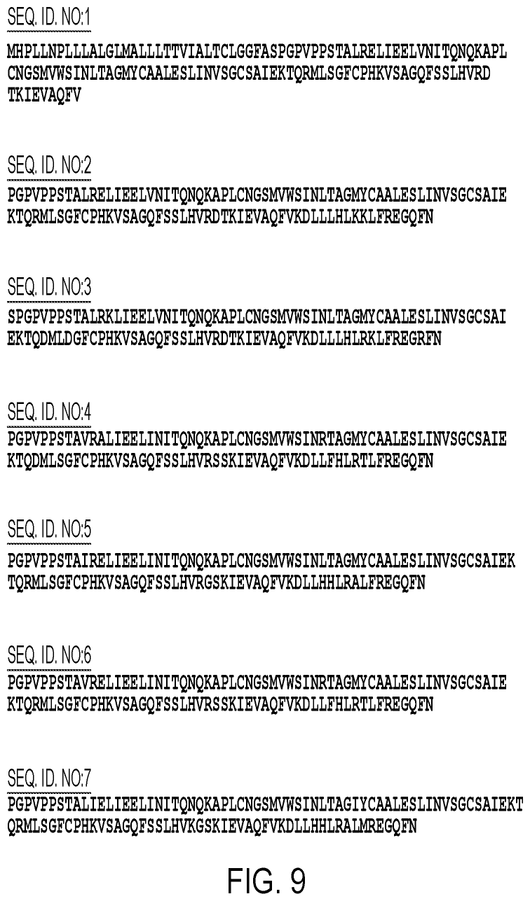

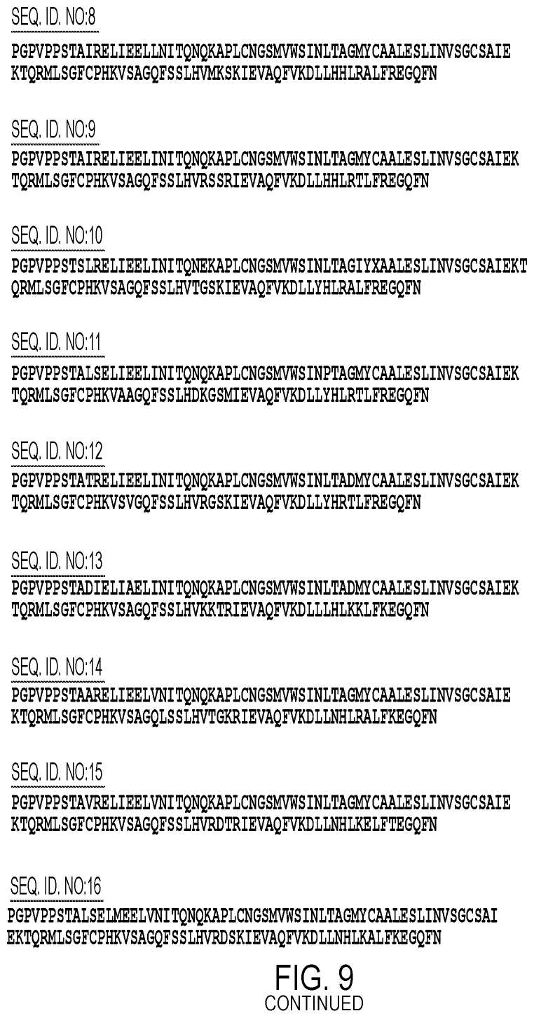

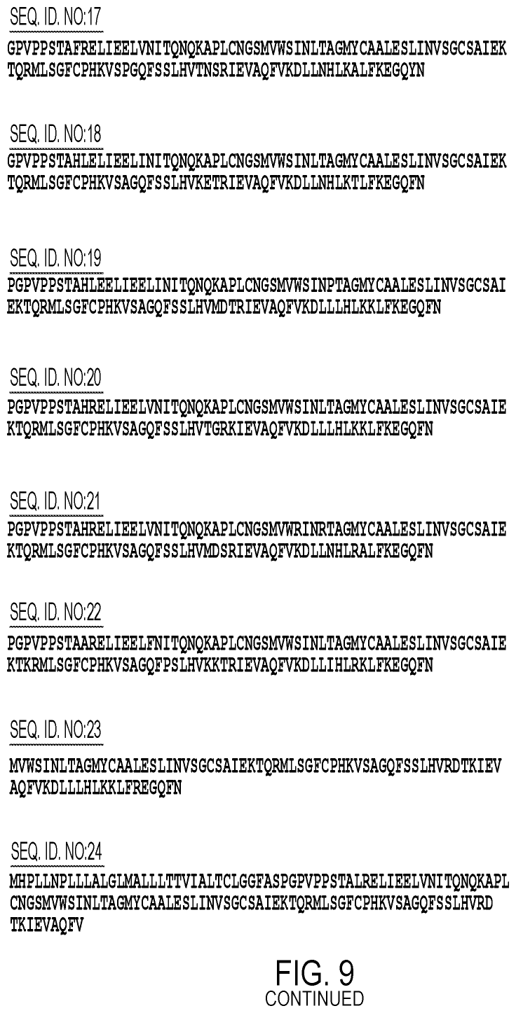

After treatment, the animals were monitored for animal survival. Results can be found in FIG. 8. As shown, treatment with cmIL13 prior to radiation substantially increased survival in the mice.

Whereas specific aspects of the invention have been described in detail, it will be appreciated by those skilled in the art that various modifications and alternatives to those details could be developed in light of the overall teachings of the disclosure. Accordingly, the particular arrangements disclosed are meant to be illustrative only and not limiting as to the scope of the invention which is to be given the full breadth of the claims appended and any and all equivalents thereof.

We claim:

1. A method of treating cancer in a subject comprising:
(i) administering a pharmaceutical composition comprising a therapeutically effective amount of a mutagenized IL13 moiety (mIL13); and then
(ii) delivering a radiation therapy.

2. The method of claim 1, wherein administering the pharmaceutical composition continues during the radiation therapy.

3. The method of claim 1, wherein administering the pharmaceutical composition is terminated before the radiation therapy begins.

4. (canceled)

5. The method of claim 1, wherein the pharmaceutical composition is administered via a catheter and/or convection enhanced delivery.

6-8. (canceled)

9. The method of claim 5, wherein the pharmaceutical composition is administered acutely.

10-11. (canceled)

12. The method of claim 5, wherein the convection enhanced delivery comprises administering the pharmaceutical composition at a flow rate of up to 2 mL/hour.

13. The method of claim 5, further comprising the step of stereostatic image-guided catheter insertion.

14. (canceled)

15. The method of claim 1, wherein the therapeutically effective dose of mIL13 is 0.03 μg/mL to 1 μg/mL.

16. The method of claim 1, wherein the method further comprises detecting expression of interleukin 13 receptor α 2 (IL13Rα2) in a sample, wherein expression of IL13Rα2 is indicative of the subject being responsive to treatment by mIL13 and radiation therapy.

17. The method of claim 1, wherein the pharmaceutical composition is administered intratumorally and/or to a resection cavity.

18. The method of claim 1, wherein the mIL13 is fused to a cytotoxin.

19. The method of claim 18, wherein the cytotoxin comprises a bacterial-derived toxin.

20. (canceled)

21. The method of claim 1, wherein the mIL13 is engineered to have increased affinity for IL13Rα2 compared to wild-type human IL13 and/or decreased affinity for interleukin 13 receptor α 1 (IL13Rα1) compared to wild-type human IL13.

22. The method of claim 1, wherein the mIL13 comprises at least one amino acid substitution as compared to SEQ ID NO:1 or SEQ ID NO: 2.

23. The method of claim 1, wherein the mIL13 comprises amino acid changes relative to wild-type IL13 at positions E13, R66, S69, and/or K105.

24. The method of claim 1, wherein the mIL13 comprises one or more amino acid substituents E13K.R66D.S69D.K105R.

25. The method of claim 1, wherein the mIL13 comprises an amino acid sequence set forth in one of SEQ ID NOS: 3 to 24 or a homologue thereof or a homologue of one of SEQ ID NO:1 or SEQ ID NO:2.

26. The method of claim 1, wherein the mIL13 differs by no more than 20 residues from SEQ ID NO: 1 or SEQ ID NO:2.

27. The method of claim 1, wherein the cancer comprises a malignant glioma.

28-29. (canceled)

30. The method of claim 1, wherein the method results in an increase in survival of the subject relative to a control subject having a malignant glioma that is not administered a pharmaceutical composition comprising a therapeutically effective amount of mIL13 and radiation therapy.


SEQUENCE LISTING (24 sequences)

SEQ ID NO: 1 - protein, 146 aa, Homo sapiens [sequence omitted]

SEQ ID NO: 2 - protein, 113 aa, Homo sapiens
PGPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGQFN

SEQ ID NO: 3 - protein, 114 aa, Homo sapiens
SPGPVPPSTALRKLIEELVNITQNQKAPLCNGSMVWSINLTAGMYCAALESLINVSGCSAIEKTQDMLDGFCPHKVSAGQFSSLHVRDTKIEVAQFVKDLLLHLRKLFREGRFN

SEQ ID NO: 4 - protein, 113 aa, Homo sapiens
PGPVPPSTAVRALIEELINITQNQKAPLCNGSMVWSINRTAGMYCAALESLINVSGCSAIEKTQDMLSGFCPHKVSAGQFSSLHVRSSKIEVAQFVKDLLFHLRTLFREGQFN

SEQ ID NO: 5 - protein, 113 aa, Homo sapiens
PGPVPPSTAIRELIEELINITQNQKAPLCNGSMVWSINLTAGMYCAALESLINVSGCSAIEKTQRMLSGFCPHKVSAGQFSSLHVRGSKIEVAQFVKDLLHHLRALFREGQFN

SEQ ID NO: 6 - protein, 113 aa, Homo sapiens
PGPVPPSTAVRELIEELINITQNQKAPLCNGSMVWSINRTAGMYCAALESLINVSGCSAIEKTQRMLSGFCPHKVSAGQFSSLHVRSSKIEVAQFVKDLLFHLRTLFREGQFN

SEQ ID NO: 7 - protein, 113 aa, Homo sapiens
PGPVPPSTALIELIEELINITQNQKAPLCNGSMVWSINLTAGIYCAALESLINVSGCSAIEKTQRMLSGFCPHKVSAGQFSSLHVKGSKIEVAQFVKDLLHHLRALMREGQFN

SEQ ID NO: 8 - protein, 113 aa, Homo sapiens
PGPVPPSTAIRELIEELLNITQNQKAPLCNGSMVWSINLTAGMYCAALESLINVSGCSAIEKTQRMLSGFCPHKVSAGQFSSLHVMKSKIEVAQFVKDLLHHLRALFREGQFN

SEQ ID NO: 9 - protein, 113 aa, Homo sapiens
PGPVPPSTAIRELIEELINITQNQKAPLCNGSMVWSINLTAGMYCAALESLINVSGCSAIEKTQRMLSGFCPHKVSAGQFSSLHVRSSRIEVAQFVKDLLHHLRTLFREGQFN

SEQ ID NO: 10 - protein, 113 aa, Homo sapiens
PGPVPPSTALRELIEELINITQNEKAPLCNGSMVWSINLTAGIYCAALESLINVSGCSAIEKTQRMLSGFCPHKVSAGQFSSLHVTGSKIEVAQFVKDLLYHLRALFREGQFN

SEQ ID NO: 11 - protein, 113 aa, Homo sapiens
PGPVPPSTALSELIEELINITQNQKAPLCNGSMVWSINPTAGMYCAALESLINVSGCSAIEKTQRMLSGFCPHKVAAGQFSSLHDKGSMIEVAQFVKDLLYHLRTLFREGQFN

SEQ ID NO: 12 - protein, 113 aa, Homo sapiens
PGPVPPSTATRELIEELINITQNQKAPLCNGSMVWSINLTADMYCAALESLINVSGCSAIEKTQRMLSGFCPHKVSVGQFSSLHVRGSKIEVAQFVKDLLYHLRTLFREGQFN

SEQ ID NO: 13 - protein, 113 aa, Homo sapiens
PGPVPPSTADIELIAELINITQNQKAPLCNGSMVWSINLTADMYCAALESLINVSGCSAIEKTQRMLSGFCPHKVSAGQFSSLHVKKTRIEVAQFVKDLLLHLKKLFKEGQFN

SEQ ID NO: 14 - protein, 113 aa, Homo sapiens
PGPVPPSTAARELIEELVNITQNQKAPLCNGSMVWSINLTAGMYCAALESLINVSGCSAIEKTQRMLSGFCPHKVSAGQLSSLHVTGKRIEVAQFVKDLLNHLRALFKEGQFN

SEQ ID NO: 15 - protein, 113 aa, Homo sapiens
PGPVPPSTAVRELIEELVNITQNQKAPLCNGSMVWSINLTAGMYCAALESLINVSGCSAIEKTQRMLSGFCPHKVSAGQFSSLHVRDTRIEVAQFVKDLLNHLKELFTEGQFN

SEQ ID NO: 16 - protein, 113 aa, Homo sapiens
PGPVPPSTALSELMEELVNITQNQKAPLCNGSMVWSINLTAGMYCAALESLINVSGCSAIEKTQRMLSGFCPHKVSAGQFSSLHVRDSKIEVAQFVKDLLNHLKALFKEGQFN

SEQ ID NO: 17 - protein, 112 aa, Homo sapiens
GPVPPSTAFRELIEELVNITQNQKAPLCNGSMVWSINLTAGMYCAALESLINVSGCSAIEKTQRMLSGFCPHKVSPGQFSSLHVTNSRIEVAQFVKDLLNHLKALFKEGQYN

SEQ ID NO: 18 - protein, 112 aa, Homo sapiens
GPVPPSTAHLELIEELINITQNQKAPLCNGSMVWSINLTAGMYCAALESLINVSGCSAIEKTQRMLSGFCPHKVSAGQFSSLHVKETRIEVAQFVKDLLNHLKTLFKEGQFN

SEQ ID NO: 19 - protein, 113 aa, Homo sapiens
PGPVPPSTAHLELIEELINITQNQKAPLCNGSMVWSINPTAGMYCAALESLINVSGCSAIEKTQRMLSGFCPHKVSAGQFSSLHVMDTRIEVAQFVKDLLLHLKKLFKEGQFN

SEQ ID NO: 20 - protein, 113 aa, Homo sapiens
PGPVPPSTAHRELIEELVNITQNQKAPLCNGSMVWSINLTAGMYCAALESLINVSGCSAIEKTQRMLSGFCPHKVSAGQFSSLHVTGRKIEVAQFVKDLLLHLKKLFKEGQFN

SEQ ID NO: 21 - protein, 113 aa, Homo sapiens
PGPVPPSTAHRELIEELVNITQNQKAPLCNGSMVWRINRTAGMYCAALESLINVSGCSAIEKTQRMLSGFCPHKVSAGQFSSLHVMDSRIEVAQFVKDLLNHLRALFKEGQFN

SEQ ID NO: 22 - protein, 113 aa, Homo sapiens
PGPVPPSTAARELIEELFNITQNQKAPLCNGSMVWSINLTAGMYCAALESLINVSGCSAIEKTKRMLSGFCPHKVSAGQFPSLHVKKTRIEVAQFVKDLLIHLRKLFKEGQFN

SEQ ID NO: 23 - protein, 81 aa, Homo sapiens
MVWSINLTAGMYCAALESLINVSGCSAIEKTQRMLSGFCPHKVSAGQFSSLHVRDTKIEVAQFVKDLLLHLKKLFREGQFN

SEQ ID NO: 24 - protein, 146 aa, Homo sapiens
MHPLLNPLLLALGLMALLLTTVIALTCLGGFASPGPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGQFN